(19) United States
(12) Patent Application Publication
(10) Pub. No.: US 2014/0135363 A1
(43) Pub. Date: May 15, 2014

(54) PROTON PUMP INHIBITOR FOR USE IN A METHOD OF TREATING DERMATOLOGICAL DISEASES IN CANINE

(71) Applicant: Joachim KARLE, Ruesselsheim (DE)

(72) Inventor: Joachim KARLE, Ruesselsheim (DE)

(73) Assignee: BOEHRINGER INGELHEIM VETMEDICA GMBH, Ingelheim am Rhein (DE)

(21) Appl. No.: 14/077,575

(22) Filed: Nov. 12, 2013

(30) Foreign Application Priority Data
Nov. 14, 2012 (EP) 12192643.0

(51) Int. Cl. A61K 31/4439

(52) U.S. Cl. CPC A61K 31/4439
USPC 514/338; 546/270.1

(57) ABSTRACT

The present invention relates to a proton pump inhibitor or a pharmaceutically acceptable salt thereof for the treatment of skin diseases in canine patients. The invention also relates to improving the quality of life, improving the general health condition as well as preventing secondary infections in canine patients suffering from skin diseases. Moreover, the present invention also relates to a pharmaceutical composition for topical administration, preferably for use in a method of treating skin diseases and/or improving the general health condition in canine patients, wherein such pharmaceutical formulation comprises a proton pump inhibitor or a pharmaceutically acceptable salt thereof and one or more excipients.

### FIELD OF THE INVENTION

The invention relates to the field of medicine, in particular to the field of veterinary medicine. The invention relates to the effect of proton pump inhibitors or a pharmaceutically acceptable salt thereof on the treatment of skin diseases, preferably any form of canine dermatoses, itchy and/or inflammatory skin diseases. According to the invention the proton pump inhibitor is administered topically. It further relates to improving the quality of life as well as the general health condition in canine patients suffering from inflammatory and/or pruitic skin diseases, in particular immune-mediated skin disorders such as for example atopic dermatitis.

### BACKGROUND OF THE INVENTION

The condition of the skin as well as the coat of canine is an important indicator of its general health. Skin disorders of dogs vary from acute, self-limiting problems to chronic or long-lasting problems requiring life-time treatment. They also need to be differentiated on the basis of being of primary or secondary (due to scratching, itch) in nature, making diagnosis complicated.

Dog skin disorders may be grouped into categories according to the causes:
i) Immune-mediated skin disorders such as e.g. canine atopic dermatitis, pemphigus foliaceus, food allergies, eczema;
ii) Physical and environmental skin diseases such as hot spot, or acute moist dermatitis, lick granulomas;
iii) Infectious skin diseases such as e.g. contagious infections including parasitic, bacterial, fungal and viral skin diseases such as canine scabies, Cheyletiella, infestation with contagious lice, or non-contagious skin infections resulting from normal bacterial or fungal skin flora that is allowed to proliferate and cause skin diseases such as Staphylococcus intermedius pyoderma and Malassezia dermatitis;
iv) Flea allergy dermatitis;
v) Hereditary and developmental skin diseases resulting in abnormalities of skin structure and function;
vi) Cutaneous manifestations of internal diseases such as e.g. endocrine abnormalities (hypothyroidism, Cushing's Syndrome, tumors) can result in skin disorders.

Many skin disorders can also be due to poor-quality food.

Thus, itchiness in dogs can be due to many skin diseases, this itchiness is being amplified by the dogs licking, biting, chewing and scratching, which may also lead to that skin infections and worsening of the itching. Without proper medication this quickly becomes a vicious cycle. Furthermore the dog's quality of life is also affected by the itchiness.

So far many skin diseases in dogs require a multifocal treatment approach, using more than one medication or treatment regimen. Treatment is further complicated by attempts to identify and eliminate the underlying cause of the condition. Often this is not possible In some cases hypersensitivity testing may be necessary to identify contact or inhalant irritants, such as house dust, mites, environmental chemicals, fertilizers, perfumes, carpet cleaners, deodorants and other household products.

However, in order to avoid any licking, biting, chewing and scratching, which amplifies the itchiness and causes escalation of the symptoms of the underlying disease process, it is crucial to stop the itchiness. Presently treatment of skin diseases may include a regimen of oral and/or topical anti-inflammatory medications to at least temporarily relieve the dog's symptoms. These may include immunosuppressant drugs, steroids or non-steroidal anti-inflammatory drugs. Sometimes oral or topical antibiotics may be prescribed if a secondary bacterial infection develops. Also antihistamines can be used for severe cases of itching; these may be oral or topical as well.

These medications, in particular steroids, can have serious adverse effects if they are given for longer periods of time. Therefore, topical administration of steroids should only be used over a short period of time to avoid side effects such as skin thinning (atrophy), pigment disorder, hypertrichosis, calcinosis cutis, and a partial suppression of the local immune system can occur. Also oral treatment has to be avoided to take place over longer periods of time due to adverse effects such as polyuria, polydipsia, systemic suppression of the immune system with resulting concomitant secondary diseases such as urinary tract infections or secondary skin infections.

Furthermore, any antibiotic treatment should only be used when absolutely necessary, as overuse of antibiotics.

So far known treatments in dogs result in undesired side-effects or do not have the desired effect. Thus, there is the need to provide a treatment of canine that improves the skin disease rapidly without any undesired side-effects and that increases the general health condition of the dog.

The problem underlying the present invention was to provide a medication, which allows the treatment of skin diseases in canine patients, in particular dogs, and/or which leads to an improved general health condition and quality of life.

### DESCRIPTION OF THE INVENTION

Before the embodiments of the present invention are discussed it shall be noted that as used herein and in the appended claims, the singular forms “a”, “an”, and “the” include plural reference unless the context clearly dictates otherwise. Thus, for example, reference to “a preparation” includes a plurality of such preparations. Unless defined otherwise, all technical and scientific terms used herein have the same meanings as commonly understood by one of ordinary skill in the art to which this invention belongs.

All given ranges and values may vary by 1 to 5% unless indicated otherwise or known otherwise by the person skilled in the art, therefore, the term “about” was omitted from the description. Although any methods and materials similar or equivalent to those described herein can be used in the practice or testing of the present invention, the preferred methods, devices, and materials are now described. All publications mentioned herein are incorporated herein by reference for the purpose of describing and disclosing the substances, excipients, carriers, and methodologies as reported in the publications which might be used in connection with the invention. Nothing herein is to be construed as an admission that the invention is not entitled to antedate such disclosure by virtue of prior invention.

The solution to the above technical problem is achieved by the description and the embodiments characterized in the claims.

The invention comprises a proton pump inhibitor or a pharmaceutically acceptable salt thereof for treating skin diseases, preferably any form of canine dermatoses or itchy and/or inflammatory skin diseases.

In accordance with some examples of the invention, the invention relates to a proton pump inhibitor or a pharmaceutically acceptable salt thereof for improving quality of life and/or improving general health condition in canine patients, in particular dogs, suffering from skin diseases, preferably any form of canine dermatoses or itchy and/or inflammatory skin diseases.

The term “skin diseases” or “skin conditions” as used herein relates to diseases of the skin, which result in itchiness. The itchy skin diseases are characterized by constant scratching, biting at the skin and rubbing up against objects to relieve the itch. Clinical manifestations are in general redness, flaky skin, hairloss blisters, crusty pustules, painful swelling, infection, inflammation and/or discharge.

There are several main indications for skin conditions, preferably any form of canine dermatoses or itchy and/or inflammatory skin diseases, whereby said conditions have different aetiologies that may result in skin diseases, such as
i) Immune-mediated skin disorders such as e.g. canine atopic dermatitis, pemphigus foliaceus, food allergies, eczema;
ii) Physical and environmental skin diseases such as Hot Spot, or acute moist dermatitis, lick granulomas;
iii) Infectious skin diseases such as e.g. contagious infections including parasitic, bacterial, fungal and viral skin diseases such as canine scabies, Cheyletiella, infestation with contagious lice, or non-contagious skin infections resulting from normal bacterial or fungal skin flora that is allowed to proliferate and cause skin diseases such as Staphylococcus intermedius pyoderma and Malassezia dermatitis;
iv) Flea allergy dermatitis;
v) Hereditary and developmental skin diseases resulting in abnormalities of skin structure and function;
vi) Cutaneous manifestations of internal diseases such as e.g. endocrine abnormalities (hypothyroidism, Cushing's Syndrome, tumors) can result in skin disorders.

Thus, in accordance with yet other examples of the invention the skin conditions are preferably itchy and/or inflammatory skin conditions, even more preferred are skin conditions preferably itchy and/or inflammatory skin conditions due to eczema, atopic dermatitis, lick granuloma, flea allergies, hot spots or food allergies.

“Dermatosis” or “dermatoses” relates to cutaneous condition, which is any medical condition that affects the organ system that comprises the entire surface of the body and includes skin, hair, nails, and related muscle and glands.

Accordingly, a further aspect of the present invention relates to a proton pump inhibitor or a pharmaceutically acceptable salt thereof for treating skin diseases, preferably any form of canine dermatoses or itchy and/or inflammatory skin diseases, more preferred skin diseases due to one or more of the above defined aetiologies. The invention also relates to a proton pump inhibitor or a pharmaceutically acceptable salt thereof for improving quality of life and/or improving general health condition in canine patients, preferably dogs, suffering from skin diseases, preferably any form of canine dermatoses or itchy and/or inflammatory skin diseases due to one or more of the above defined aetiologies.

Canine patients, in particular dogs, become increasingly submissive, anxious, and snappy when suffering from skin diseases, especially itchy skin diseases due to the constant scratching. The term “quality of life” as used herein relates to a tendency of a better quality of life (QoL) following treatment with a proton pump inhibitor, as the treatment of the skin disease with the proton pump inhibitors results in a significant improvement of itchiness. The assessment of the increased quality of life is mainly based on the pet owner and includes an evaluation of behavior in general with regard to its activity level, personality, and itchiness.

The term “improving general health condition” as used herein relates to improvement of the actual state of the skin disease and the condition of the fur coat of the canine patient, in particular a dog. Furthermore, an improvement is anticipated as the treated skin is not prone to infections or inflammations anymore due to less scratching and biting of the diseased skin. Thus improving the general health condition also relates to preventing secondary skin infections such as inflammation. Therefore the invention also relates to a pharmaceutical composition comprising a proton pump inhibitor or a pharmaceutically acceptable salt thereof for the prevention of secondary infections in canine suffering from skin diseases as defined above.

“Proton pump inhibitors” relate to a proton pump inhibitor including pharmaceutically acceptable salts thereof, which act by irreversibly blocking the hydrogen/potassium adenosine triphosphatase enzyme system (the H⁺/K⁺ATPase) of the gastric parietal cells. The proton pump is the terminal stage in gastric acid secretion, being directly responsible for secreting H⁺ ions into the gastric lumen, making it an ideal target for inhibiting acid secretion. Proton pump inhibitors are usually used for the treatment of gastric ulceration. However, it has been surprisingly found that these compounds result in a quick relief in itchiness. The invention therefore also relates to the use of a proton pump inhibitor for the treatment of treating skin diseases, preferably any form of canine dermatoses or itchy and/or inflammatory skin diseases, even more preferred skin diseases due to one or more of the above defined aetiologies.

Preferred proton pump inhibitors are inhibitors are omeprazol, esomeprazole, lansoprazol or pantoprazol or a pharmaceutically acceptable salt of any of these compounds. Preferred is omeprazol or a pharmaceutically acceptable salt thereof.

The term “patient” as used herein relates to canine patients, preferably dogs, even more preferred domestic dogs, in particular dogs suffering from skin diseases, even more preferred to dogs suffering from itchy skin diseases. According to another embodiment of the invention the canine patients suffer skin diseases due to one or more of the above defined aetiologies.

Thus, according to one aspect of the invention, a proton pump inhibitor or a pharmaceutically acceptable salt thereof, in particular omeprazol, esomeprazole, lansoprazol or pantoprazol, preferably omeprazol is used for treating skin diseases, preferably any form of dermatoses or itchy and/or inflammatory skin diseases in canine patients, preferably dogs. Thus according to the invention the proton pump inhibitors or a pharmaceutically acceptable salt thereof, in particular omeprazol, esomeprazole, lansoprazol or pantoprazol, preferably omeprazol is used for treating skin diseases, preferably the treatment of treating skin diseases, preferably any form of dermatoses or itchy and/or inflammatory skin diseases due to one or more of the above defined aetiologies.

According to one aspect of the invention the canine patient, preferably a dog, is additionally monitored with regard to its overall well-being. Administration of a proton pump inhibitor, in particular omeprazol, to a canine patient, in particular to dogs, results in not just the treatment of the skin diseases or any form of dermatosis or itchy and/or inflammatory skin diseases as defined above but also to an improved quality of life and/or general health condition.

Thus, according to a further aspect of the invention it is found by the inventors that the use of a proton pump inhibitor or a pharmaceutically acceptable salt thereof, in particular omeprazol, esomeprazol, lansoprazol or pantoprazol, preferably omeprazol, improves the quality of life of canine patients, in particular dogs, preferably suffering from skin diseases, preferably any form of dermatoses or itchy and/or inflammatory skin diseases due to one or more of the above defined aetiologies.

Furthermore, the inventors have found that a proton pump inhibitor or a pharmaceutically acceptable salt thereof, in particular omeprazol, esomeprazol, lansoprazol or pantoprazol, preferably omeprazol, also improves the general health condition of canine patients, in particular dogs, preferably suffering from skin diseases, preferably any form of dermatoses or itchy and/or inflammatory skin diseases due to one or more of the above defined aetiologies.

### Administration

Generally a topical application has fewer side effects than a systemic administration, since a high concentration of active pharmaceutical compound is present only in the corresponding target area. Thus, the risks of any side effects with the above defined proton pump inhibitors, which are known for the systemic administration, are negligible for topical application.

Thus, according to exemplary embodiments of the invention, the administration of a proton pump inhibitor, as defined above, is topical to an animal suffering from skin diseases, preferably any form of dermatoses or itchy and/or inflammatory skin diseases due to one or more of the following aetiologies:
i) Immune-mediated skin disorders such as e.g. canine atopic dermatitis, pemphigus foliaceus, food allergies, eczema;
ii) Physical and environmental skin diseases such as Hot Spot, or acute moist dermatitis, lick granulomas;
iii) Infectious skin diseases such as e.g. contagious infections including parasitic, bacterial, fungal and viral skin diseases such as canine scabies, Cheyletiella, infestation with contagious lice, or non-contagious skin infections resulting from normal bacterial or fungal skin flora that is allowed to proliferate and cause skin diseases such as Staphylococcus intermedius pyoderma and Malassezia dermatitis;
iv) Flea allergy dermatitis;
v) Hereditary and developmental skin diseases resulting in abnormalities of skin structure and function;
vi) Cutaneous manifestations of internal diseases such as e.g. endocrine abnormalities (hypothyroidism, Cushing's Syndrome, tumors).

According to exemplary aspects of the invention, the administration of a proton pump inhibitor as defined above is topical to an animal suffering from itchy and/or inflammatory skin conditions, even more preferred are skin conditions preferably itchy and/or inflammatory skin conditions due to eczema, atopic dermatitis, lick granuloma, flea allergies, hot spots or food allergies.

According to yet another aspect of the invention, the pharmaceutical composition can be a single-or multi-phase system, meaning homogeneous systems or emulsions, depending on the type of excipients that are used. Depending on the excipients the pharmaceutical composition can be ointments, creams, gels, emollients, spray formulations, lotions or the like.

The pharmaceutical composition according to one aspect the invention is advantageously intended for topical application, i.e. for external application on the affected areas of the skin.

The pharmaceutical composition may be, in exemplary embodiments, single-phase or multi-phase systems, including homogeneous systems or emulsions which, depending on the type of basic substances or carrier substances used, are called ointments, creams, gels or lotions. According to a preferred embodiment of the invention, the pharmaceutical composition is present in the form of an ointment that is as a single-phase system with a lipophilic carrier substance.

The pharmaceutical composition comprises a proton pump inhibitor or the pharmaceutically acceptable salt thereof in a concentration of 0.1 to 20 wt %, preferably 0.2 to 15 wt %, 0.3 to 10 wt %, 0.4 to 10 wt %, 0.5 to 10 wt %, 0.6 to 10 wt %, 0.7 to 10 wt %, 0.8 to 10 wt %, 0.9 to 10 wt %, 1 to 10 wt %, 2 to 9 wt %, 3 to 8 wt %, 4 to 7 wt %, 5 to 6 wt %, even more preferred 1 wt %, 5wt % and 10 wt %.

In some embodiments of the invention, it is advantageous if pharmaceutical compositions comprise a lipophilic carrier substance as a basic ointment substance. The lipophilic carrier substance is preferably selected from synthetic and mineral waxes, fats and oils and synthetic derivatives thereof, as well as mixtures thereof. Examples of synthetic or mineral carrier substances are polyethylene glycols, aliphatic hydrocarbons (for example various paraffins and petroleum jelly) and silicones, which are in each case available in different molecular weight ranges.

Alternatively or additionally, pharmaceutical compositions may also comprise a lipophilic carrier substance, which is selected from animal and vegetable waxes, fats and oils and their hydrogenated or partially hydrogenated derivatives, as well as mixtures thereof, such as for example beeswax, lanolin or plant oils.

In some embodiments the pharmaceutical composition can be anhydrous, preferably substantially anhydrous, as the pyridylmethylsulfinyl benzimidazoles, such as the above defined proton pump inhibitors, are generally acid-sensitive as well as water-sensitive. By using anhydrous systems in exemplary embodiments of the invention, the stability and durability of the pharmaceutical composition can therefore be increased.

However, it has been found that the pharmaceutical composition according to other embodiments of the invention can be formulated as a two-phase system with a water fraction of up to 40 wt % with an adequate stability. According to this further preferred embodiment of the invention the pharmaceutical composition, is a water-in-oil emulsion or an oil-in-water emulsion. In this exemplary case, the composition may also comprise one or more emulsifiers.

By adding suitable auxiliary substances, the acid-sensitivity of the proton pump inhibitors can additionally be taken into consideration. In order to adjust a pH of the carrier substance used in the alkaline range, preferably with a pH between 8 and 10, even more preferred from 8.5 to 9.5, the pharmaceutical composition advantageously comprises an alkaline substance such as for example an alcoholic potassium hydroxide solution, preferably in a quantity of 0.1 to 5 wt %.

The pharmaceutical composition according to one aspect of the invention may also comprise one or more buffering agents, preferably in a quantity of up to 5 wt %. This may be an anhydrous composition, in particular lipophilic amines, such as, for example stearylamine, which are soluble in a lipophilic carrier substance and are used to buffer protons, or in a two-phase system, for example, trometamol.

To bind any residual water that may be in an anhydrous pharmaceutical composition, the latter may comprise silicon dioxide, preferably in a quantity of 0.1 to 3 wt %.

In order to prevent oxidative destruction of the proton pump inhibitor and to increase the stability of the pharmaceutical composition, the latter may additionally comprise one or more lipophilic antioxidants, preferably in a quantity of up to 5 wt %, in particular from 0.01 to 2 wt %. Suitable lipophilic antioxidants are, for example, vitamin E, butylhydroxytoluene (BHT), butylhydroxyanisole (BHA) and ascorbyl palmitate.

In order to ensure an alkaline environment in the pharmaceutical composition, alternatively or in addition to the above-described measures, it is advantageous if a conditioning with an alkaline medium is carried out.

Exemplary embodiments of the present invention therefore also comprise a method for producing a pharmaceutical composition, comprising a proton pump inhibitor, for topical application, wherein the proton pump inhibitor is mixed with a lipophilic carrier substance and this is then treated with an alkaline medium. For example, a pharmaceutical composition, can be an ointment with the lipophilic carrier substance as the basic ointment substance, as was described above.

The alkaline medium, with which the lipophilic carrier substance is treated, preferably comprises an aqueous alkaline solution, in other words, for example, an aqueous sodium hydroxide solution or potassium hydroxide solution. Furthermore, the alkaline medium may comprise one or more stabilizers and/or antioxidants, such as, for example, sodium disulfide or sodium EDTA.

It is favorable if the lipophilic carrier substance is boiled out with the alkaline medium. The alkaline medium is then preferably removed.

The above-described auxiliary substance(s) can be added to the lipophilic carrier substance before or after the treatment with the alkaline medium.

Thus, one exemplary embodiment of the invention provides a pharmaceutical composition for topical administration for use in a method of treating skin diseases and/or improving quality of life and/or improving general health condition in canine patients, wherein the pharmaceutical formulation comprises a proton pump inhibitor or a pharmaceutically acceptable salt thereof and one or more excipients selected from one or more carrier, one or more antioxidant, and/or one or more buffering agent. Preferably the pharmaceutical composition comprises a proton pump inhibitor or a pharmaceutically acceptable salt thereof, one or more lipophilic carrier, one or more lipophilic antioxidants and/or one or more lipophilic buffering agent.

According to another aspect of the invention, the pharmaceutical composition may be an anhydrous system containing a proton pump inhibitor or a pharmaceutically acceptable salt thereof, preferably omeprazole, one or more lipophilic carriers, preferably petroleum jelly, Plastibase DAC, medium-chain triglycerides and beeswax, various paraffins, such as paraffin oil, paraffin subliquidum, hard paraffin, and one or more antioxidants such as vitamin E and/or butylhydroxytoluene, and optionally lipophilic buffering agent such as stearylamine.

According to yet another exemplary embodiment of the invention, the pharmaceutical composition may also be a water-in-oil emulsion containing a proton pump inhibitor or a pharmaceutically acceptable salt thereof, preferably omeprazole, one or more lipophilic carriers such as lanolin, petroleum jelly, medium-chained triglycerides, various paraffins, preferably paraffin subliquidum, one or more lipophilic antioxidants such as vitamin E, butylhydroxytoluene, butylhydroxyanisole or ascorbyl palmitate, or one or more buffering agents such as lipophilic amines, preferably trometamol.

Thus, the pharmaceutical composition according to one aspect of the invention is for use in the treatment of skin diseases, preferably any form of dermatoses or itchy and/or inflammatory skin diseases due to any of the above defined aetiologies in canine patients, preferably dogs.

The method for producing the pharmaceutical composition can be carried out completely or partially under a protective gas atmosphere, for example nitrogen or argon. An oxidization of the proton pump inhibitor can thereby be avoided or at least reduced. In particular, the cooling of the lipophilic carrier substance after boiling out with the alkaline medium and the further processing preferably take place under a protective gas atmosphere.

Filling the pharmaceutical composition is also preferably carried out under a protective gas atmosphere. Suitable containers comprise, in particular, tubes made of aluminum, plastics material or a composite material, for example aluminum tubes with a septum, polyfoil tubes (plastics material/aluminum/plastics material), multiplex composite tubes or plastics material containers produced by the blow-seal method.

The tubes or other containers for the pharmaceutical composition may have an inner coating, which contains antioxidants as additional product protection.

According to a further embodiment of the invention, as an alternative form of administration of the pharmaceutical composition, it may also be provided that the proton pump inhibitor or the composition is integrated into a polymer film, which is applied to the affected point on the skin. It is particularly advantageous here if the active ingredient is continuously dispensed from the polymer film.

### Formulations

Various pharmaceutical compositions are produced according to exemplary embodiments of the present invention (preparations 1 to 4) in the form of ointments. For example, in one embodiment compositions contain, as the proton pump inhibitor, micronized omeprazole, and various mixtures of lipophilic carrier substances as the basic ointment substance (petroleum jelly, various paraffins, Plastibase DAC, medium-chain triglycerides and beeswax). Furthermore, the compositions contain oil-soluble vitamin E and butylhydroxytoluene as lipophilic antioxidants and optionally stearylamine as the lipophilic buffering agent.

The precise composition of the formulations 1 to 4 is given in the following Table 1, in which all the data are given in wt %.

TABLE 1

| | Formulation 1 | Formulation 2 | Formulation 3 | Formulation 4 |
|---|---|---|---|---|
| Omeprazole | 1.00 | 1.00 | 1.00 | 1.00 |
| Petroleum Jelly | 82.98 | 72.98 | 82.98 | |
| Paraffin | 5.00 | | | |
| Subliquidum | | | | |
| Paraffin Oil | | | 10.00 | 9.00 |
| Hard Paraffin | | | 5.00 | |
| Plasticbase DAC | | | | 88.98 |
| Medium-chain | 5.00 | 15.00 | | |
| Triglycerides | | | | |
| Beeswax | 5.00 | 8.00 | | |
| Vitamin E | 1.00 | 1.00 | 1.00 | 1.00 |
| Butylhydroxytoluene | 0.02 | 0.02 | 0.02 | 0.02 |
| Stearylamine | | 2.00 | | |

Further examples of antioxidants are butylhydroxyanisole (0.01 to 0.5 wt %) and ascorbyl palmitate (0.01 to 0.2 wt %).

According to the exemplary embodiment of the present invention the pharmaceutical composition (Formulation 5) is produced in the form of a two-phase system (water-in-oil emulsion). The precise composition is given in Table 2. All data are given in wt %.

TABLE 2

| | Formulation 5 |
|---|---|
| Paraffin Subliquidum | 5.0 |
| Lanolin | 8.0 |
| Petroleum Jelly | 40.0 |
| Medium-chain Triglycerides | 7.5 |
| Vitamin E Acetate | 5.0 |
| Omeprazole | 1.0 |
| Butylhydroxytoluene | 0.2 |
| Water | 33.0 |
| Trometamol | 0.3 |

For production, the lipophilic carrier substances (paraffin subliquidum, lanolin, petroleum jelly and the medium-chain triglycerides) are melted at 80 degree Celsius. Trometamol is dissolved in the water and incorporated in the lipophilic carrier substances and homogenized. After cooling to about 25 to 30 degree Celsius, omeprazole is dissolved or suspended in the vitamin E acetate, and butylhydroxytoluene is incorporated.

According to the present invention the pharmaceutical composition (Formulation 6) is produced in the form of an ointment. The precise composition is given in Table 3. All data are given in wt %.

TABLE 3

| | Formulation 6 |
|---|---|
| Paraffin Subliquidum | 7.5 |
| Lanolin | 24.0 |
| Petroleum Jelly | 51.3 |
| Medium-chain Triglycerides | 5.0 |
| Vitamin E Acetate | 2.0 |
| Omeprazole | 5.0 |
| Water | 4.0 |
| Trometamol | 1.2 |

Alternatively the active ingredient may be incorporated into a plaster or polymer film that can be placed on the affected skin. This is particularly useful when doing a continuous drug release from the polymer film takes place.

According to one exemplary embodiment of the invention the pharmaceutical composition comprising a proton pump inhibitor or a pharmaceutically acceptable salt thereof for topical administration for the treatment of skin diseases, preferably any form of dermatoses or itchy and/or inflammatory skin diseases as defined above for canine patients, preferably itchy and/or inflammatory skin conditions due to eczema, atopic dermatitis, lick granuloma, flea allergies, hot spots or food allergies.

The pharmaceutical composition comprising the proton pump inhibitor or a pharmaceutically acceptable salt thereof, as defined above can be administered once, twice or three times per day, preferably once or twice, even more preferred once per day. The frequency of the application on the affected skin depends, however, on the severity of the disease.

The duration of the treatment with the pharmaceutical composition comprising a proton pump inhibitor for the treatment of skin diseases, preferably any form of dermatoses or itchy and/or inflammatory skin diseases as defined above in canine patients, preferably dogs, depends on the severity of the disease. Therefore, the period of treating said diseases can be from 1 to 21 days, preferably 1 to 14 days, 1 to 10 days, 1 to 7 days, 1 to 5 days or 3 days. These periods of treatment should not be regarded as limiting as the treatment can take less than 2 days or longer than 21 days depending on the skin disease as well as on the severity of the skin disease. Many of the above defined skin diseases in canine can be chronic, which means that the skin condition is permanent or that the skin disease appears in regular or irregular periods. The duration of the treatment can start with first signs of symptoms until these completely disappear or the treatment can be continuous or without any time limits for preventing any flare ups.

Thus exemplary embodiments of the invention also provide the use of a proton pump inhibitor or a pharmaceutically acceptable salt thereof for preparing a medicament for the treatments as described herein, wherein the proton pump inhibitor or a pharmaceutically acceptable salt thereof is selected from, in particular omeprazol, esomeprazol, lansoprazol or pantoprazol, preferably omeprazol.

Also, exemplary embodiments of the invention further provide for the use of a proton pump inhibitor or a pharmaceutically acceptable salt thereof for preparing a medicament for exemplary treatments as described herein, wherein the proton pump inhibitor or the pharmaceutically acceptable salt thereof is contained in a pharmaceutical composition in overall liquid form e.g. ointment, emollient, crème, lotion and the like, comprising the proton pump inhibitor or the pharmaceutically acceptable salt thereof in a concentration of 0.1 to 20 wt %, preferably 0.2 to 15 wt %, 0.3 to 10 wt %, 0.4 to 10 wt %, 0.5 to 10 wt %, 0.6 to 10 wt %, 0.7 to 10 wt %, 0.8 to 10 wt %, 0.9 to 10 wt %, 1 to 10 wt %, 2 to 9 wt %, 3 to 8 wt %, 4 to 7 wt %, 5 to 6 wt %, even more preferred 1 wt %, 5 wt % and 10 wt %.

Embodiments of the invention thus also provide a method of
treating skin diseases as defined above and/or
improving the quality of life and/or
improving general health condition in a canine patient, preferably a dog, wherein the proton pump inhibitor is selected from omeprazol, esomeprazol, lansoprazol or pantoprazol, preferably omeprazol. Furthermore the method also prevents secondary skin infections, such as inflammation in the diseased skin areas of the canine patient.

Also, embodiments of the invention provide a method of
treating skin diseases as defined above and/or
improving the quality of life and/or
improving general health condition in a canine patient, preferably a dog, as described herein, wherein the proton pump inhibitor or the pharmaceutically acceptable salt thereof is contained in a pharmaceutical composition in overall liquid form, e.g. ointment, emollient, crème, lotion and the like, comprising the proton pump inhibitor or the pharmaceutically acceptable salt thereof in a concentration of 0.1 to 20 wt %, preferably 0.2 to 15 wt %, 0.3 to 10 wt %, 0.4 to 10 wt %, 0.5 to 10 wt %, 0.6 to 10 wt %, 0.7 to 10 wt %, 0.8 to 10 wt %, 0.9 to 10 wt %, 1 to 10 wt %, 2 to 9 wt %, 3 to 8 wt %, 4 to 7 wt %, 5 to 6 wt %, even more preferred 1 wt %, 5wt % and 10 wt %.

### EXAMPLES

The following examples serve to further illustrate exemplary embodiments of the present invention; but the same should not be construed as a limitation of the scope of the invention disclosed herein.

### Example 1

### Treatment of Dogs Suffering from Skin Diseases

Five dogs, different breeds, suffering from different skin conditions such as hot spots, atopic dermatitis and eczema, have treated with a pharmaceutical composition according to the invention containing 1% of omeprazole. The pharmaceutical composition was applied onto the affected skin once or twice per day.

In most dogs the skin condition completely disappeared after 5 days of treatment with 1 wt % or 5 wt % omeprazole. In more severe cases, i.e. dogs with several affected areas the, the condition improved significantly (table 4).

TABLE 4

| | wt % of | | | Duration of | |
|---|---|---|---|---|---|
| | omeprazole | Dog Breed | Diagnose | Therapy | Success of Therapy |
| Dog 1 | 1% | Westhighland Terrier | Atopic Dermatitis, several | 7 days | good |
| | | | affected areas | | |
| Dog 2 | 1% | Mixed breed | Hot spots | 5 days | healed |
| Dog 3 | 1% | Westhighland Terrier | Dermatitis, several affected | 7 days | improved |
| | | | areas | | |
| Dog 4 | 1% | Mixed Breed | Eczema between toes | 5 days | healed |
| Dog 5 | 5% | Labrador | Eczema between toes | 5 days | healed |

What is claimed is:

1. A proton pump inhibitor or a pharmaceutically acceptable salt thereof for use in a method of treating skin diseases in canine patients.

2. The proton pump inhibitor or a pharmaceutically acceptable salt thereof for use in the method according to claim 1 wherein the canine patient is a dog.

3. The proton pump inhibitor or a pharmaceutically acceptable salt thereof for use in a method according to claim 2, wherein the canine patients suffer from itchy skin diseases, preferably any form of canine dermatoses or itchy and/or inflammatory skin diseases, more preferably itchy and/or inflammatory skin conditions due to eczema, atopic dermatitis, lick granuloma, flea allergies, hot spots or food allergies.

4. The proton pump inhibitor or a pharmaceutically acceptable salt thereof for use in a method according to claim 1 , wherein the proton pump inhibitor is omeprazol, esomeprazol, lansoprazol or pantoprazol, preferably omeprazol.

5. The proton pump inhibitor or a pharmaceutically acceptable salt thereof for use in a method according to claim 1, wherein the proton pump inhibitor or a pharmaceutically acceptable salt thereof is formulated in topical form, preferably as an ointment.

6. The proton pump inhibitor or a pharmaceutically acceptable salt thereof for use in a method according to claim 1, wherein the proton pump inhibitor or a pharmaceutically acceptable salt thereof is to be administered once, twice or three times per day.

7. The proton pump inhibitor or a pharmaceutically acceptable salt thereof for use in a method according to claim 1, wherein the concentration of the proton pump inhibitor is 0.1 wt % to 20 wt %.

8. A pharmaceutical composition for topical administration for use in a method of treating skin diseases in canine patients, wherein such pharmaceutical formulation comprises a proton pump inhibitor or a pharmaceutically acceptable salt thereof and one or more excipients.

9. The pharmaceutical composition for use in a method according to claim 8, wherein said excipients contain one or more carriers, one or more antioxidants, and/or one or more buffering agents.

10. The pharmaceutical composition according to claim 8, for use in a method of treating skin wherein the canine patient is a dog.

11. The pharmaceutical composition for use in a method according claim 8, wherein the canine patients suffer from itchy skin diseases, preferably any form of canine dermatoses or itchy and/or inflammatory skin diseases, more preferably itchy and/or inflammatory skin conditions due to eczema, atopic dermatitis, lick granuloma, flea allergies, hot spots or food allergies.

12. The pharmaceutical composition for use in a method according to claim 8, wherein the proton pump inhibitor is omeprazol, esomeprazol, lansoprazol or pantoprazol, preferably omeprazol.

13. The pharmaceutical composition for use in a method according to claim 8, wherein the pharmaceutical composition is formulated in topical form, preferably as an ointment.

14. The pharmaceutical composition for use in a method according to claim 8, wherein the pharmaceutical composition is to be administered once, twice or three times per day.

15. The pharmaceutical composition for use in a method according to claim 8, wherein the concentration of the proton pump inhibitor is 0.1 wt % to 20 wt %.
